(19) United States
(12) Patent Application Publication
(10) Pub. No.: US 2009/0199326 A1
(43) Pub. Date: Aug. 13, 2009

(54) RETRACTABLE EARPLUG ASSEMBLY FOR A HARDHAT

(75) Inventors: Bill E. Brauner, Angels Camp, CA (US); James E. Duffy, Tahoe City, CA (US); Eivind Clausen, Bellingham, WA (US)

Correspondence Address: Beyer Law Group LLP, P.O. BOX 1687, Cupertino, CA, 95015-1687, US

(73) Assignee: StreamWorks, Inc., Stockton, CA (US)

(21) Appl. No.: 12/366,361

(22) Filed: Feb. 5, 2009

Related U.S. Application Data
(60) Provisional application No. 61/149,679, filed on Feb. 3, 2009.
(60) Provisional application No. 61/189,745, filed on Aug. 21, 2008.
(60) Provisional application No. 61/063,769, filed on Feb. 5, 2008.

(51) Int. Cl. A42B 1/06; A61F 11/08

(52) U.S. Cl. 2/423; 181/135

(57) ABSTRACT

A retractable earplug assembly is provided that is configured to retrofit to a hardhat that includes a protective housing removably mountable to the hardhat. The earplug assembly further includes a guide base slideably cooperating with a guide portion of the housing for movement thereof along a fixed path. The earplug assembly includes an elongated flexible tether having a distal portion thereof mounted to an earplug device, and a proximal portion thereof mounted to the guide base. When the guide base is selectively moved along the fixed path, the tether and the earplug device move between a retracted condition and an extended condition, enabling usable access to the earplug device.

### RELATED APPLICATIONS

This application claims priority under 35 U.S.C. 119(e) from co-pending U.S. Provisional Patent Application No. 61/149,679, filed Feb. 3, 2009, entitled “RETRACTABLE EARPLUG ASSEMBLY FOR A HARDHAT, naming Brauner et al. as inventors, and which is incorporated herein by reference in its entirety and for all purposes. This application further claims priority from co-pending U.S. Provisional Patent Application No. 61/189,745, filed Aug. 21, 2008; and further claims priority from co-pending U.S. Provisional Patent Application 61/063,769, filed Feb. 5, 2008; both titled “RETRACTABLE EARPLUG RETROFIT KIT ASSEMBLY” and naming Brauner et al. as inventors, and both of which are incorporated herein by reference in their entirety and for all purposes.

### FIELD OF THE INVENTION

The present invention relates to earplug devices, and more particularly, relates to retractable tether mounted earplug assemblies retrofitted to head wear apparel, such as hardhat.

### BACKGROUND OF THE INVENTION

It is well known that repeated or prolonged exposure to insalubrious sounds of sufficiently high sound pressure level (SPL) may cause temporary or permanent hearing loss. This is never more apparent than at building construction sites where jackhammers, drilling, and catcalls are sometimes heard simultaneously. Alone, each task can generate a significant sound level, but two or more tasks performed simultaneously can be very detrimental to those close to either event.

Fortunately, earplugs and ear protection devices have been developed that prevent harm to the eardrum and allow individuals to endure extended time in such environments. More recently, high fidelity earplugs have been developed that can be tuned to isolate the ear from certain ranges of high or low frequencies.

While the vast majority of construction workers apply some form of ear protection voluntarily, such ear protection is often mandated by state law or by the construction company itself. Typically, construction workers merely place such ear protection in their pockets or other retrievable region, during nonuse, pulling them out for use when necessary. Such small earplugs, however, are easily lost or misplaced since they often only measure less than an inch in length,

Accordingly, there is a need for a device retractably mounting earplugs to a headwear device, preferably a hardhat.

### SUMMARY OF THE INVENTION

The present invention provides a retractable earplug assembly for a hardhat. Briefly, the earplug assembly includes an elongated protective housing, a mounting assembly, a guide base and earplug and an elongated flexible tether. The protective housing defines an interior channel extending generally from one end of the housing to an opposite end thereof, and further defines an elongated guide portion, extending substantially adjacent the interior channel, and an elongated mount support portion. The housing is removably mounted to the interior wall of the hardhat through the mounting assembly which is configured to cooperate with the mount support portion and the interior facing wall of the crown portion for removably mounting therebetween. The guide base slideably cooperates with the guide portion for movement thereof along a fixed path in a direction generally longitudinally along substantially adjacent the interior channel between a first position and a second position. A distal portion of the elongated flexible tether is mounted to the earplug device, and a proximal portion thereof is mounted to the guide base. Hence when the guide base moves along the fixed path between the first position and the second position, the tether and the earplug device move between a retracted condition and an extended condition, enabling usable access to the earplug device.

In one specific embodiment, the mounting assembly comprises a VELCRO®-type fastener material.

In another specific arrangement, the guide portion and the guide base cooperate to form a zipper-type mechanism.

In still another embodiment, the protective housing is composed of a flexible sheet material.

Another specific embodiment provides a protective housing that is configured to cooperate with the hardhat crown portion such that when removably mounted thereto, the opposite end is position such that the earplug device is positioned proximate the bottom edge portion of the hardhat when in the retracted condition.

In yet another specific embodiment, the protective housing is comprised of a moldable polymer material. Further, the support portion is of a curvature generally conforming to that of the interior wall of the hardhat crown portion that the earplug assembly is to be mounted to.

In another embodiment, the elongated support portion of the housing is configured to seat in an elongated OEM channel formed in the interior wall of the hardhat.

Yet, another specific arrangement provides a mounting assembly that includes a plurality of spaced tab devices configured for friction fit mounting of the protective housing to the crown portion interior wall. The tab devices are disposed in pairs on opposite sides of the OEM channel at two or more spaced locations longitudinally therealong. Further, each tab device includes a tab member upstanding from the hardhat interior wall, and a corresponding engagement indent formed in aligned locations along the support portion. The indents are configured for aligned engagement with a corresponding tab member when the protective housing is mounted to the crown portion.

In still another embodiment, the housing guide portion defines a pair of opposed elongated flange portions forming an elongated slot extending along and in communication with the interior channel. The guide base slideably cooperates with the opposed flange portions for sliding movement between the first position and the second position.

Another arrangement includes a covered parking structure that cooperates with the hardhat crown portion to form a covered parking receptacle. This parking structure is formed and dimensioned for receipt of the earplug device therein in a manner substantially concealing the earplug device under the parking structure when in the retracted position.

In another aspect of the present invention, a hardhat assembly is provided that includes a hardhat having a crown portion with an interior facing wall, and a bottom edge portion thereof configured to extend generally equatorially around a forehead of a user when the hardhat is retained on the user's head. The assembly includes an elongated protective housing extending along the interior facing wall, and defines an interior channel extending generally from one end of the housing to an opposite end thereof. The housing further defines an elongated guide portion, extending substantially adjacent the interior channel. A guide base is provided that slideably cooperates with the guide portion for movement thereof along a fixed path in a direction generally longitudinally along substantially adjacent the interior channel between a first position and a second position. An elongated flexible tether is included having a distal portion thereof mounted to an earplug device, and a proximal portion thereof mounted to the guide base such that when the guide base moves along the fixed path between the first position and the second position, the tether and the earplug device move between a retracted condition and an extended condition, enabling usable access to the earplug device.

In one specific embodiment, the protective housing is integrally molded with the interior facing wall of the crown portion.

Another specific arrangement, a protective housing is independent of the hardhat, and includes a support portion and a mounting assembly. The mounting assembly is configured to cooperate with the support portion and the interior facing wall of the crown portion to removably mount the protective housing thereto.

In another embodiment, the hardhat assembly further includes a second earplug device, and an elongated flexible second tether having a distal portion thereof mounted to the second earplug device. A proximal portion of the second tether is mounted to the guide base such that when the guide base moves along the fixed path between the first position and the second position, the first and second tether and the first and second earplug device move simultaneously between the retracted condition and the extended condition, enabling usable access to the first and second earplug devices.

In accordance with another aspect of the present invention, a hardhat assembly is provided that include a hardhat and a retractable earplug assembly. The Earplug assembly includes a protective housing defining a mount support portion, a tether support structure, a first earplug device, and an elongated flexible first tether. This tether includes a distal portion mounted to the first earplug device, and a proximal portion thereof movably cooperating with the tether support structure between a first position and a second position. In the first position of the first tether proximal portion, the elongated first tether retractably cooperates with the tether support structure such that the distal portion of the flexible first tether and the first earplug device are positioned proximate to the protective housing, in a retracted condition. In the second position, the distal portion of the flexible first tether and the first earplug device can be repositioned away from the protective housing by a selected distance, enabling usable access to the first earplug device, in an extended condition. Finally, a mounting assembly is configured to cooperate with the mount support portion and a surface of the hardhat to removably mount the protective housing thereto.

In one specific embodiment, the tether support structure includes a first reel mechanism having a first spool rotating about a first spool axis. The proximal portion of the first tether cooperates with the first spool for rotation thereof about the first spool axis between the first position and second position. The first reel mechanism includes a first torsion spring cooperating with the first spool to bias the spool toward the first position. Consequently, the first tether and the first earplug assembly are biased toward the retracted condition.

In another embodiment, the first reel mechanism is configured to spool out the first tether toward the extended condition, in a stepped manner. Thus, the rotation of the first spool toward the first position and rewinding of the first tether toward the retracted condition is prevented. The first reel mechanism therefore includes a release device, selectively engagable to enable the first torsion spring to rotate the first spool toward the first position.

Still another specific embodiment provides a second earplug device; and an elongated flexible second tether having a distal portion thereof mounted to the second earplug device. A proximal portion of the second tether cooperates with the first spool for rotation about the first spool axis between the first position and second position. The first tether and the second tether, as a result, simultaneously move between the retracted condition and the extended condition.

In another specific embodiment, the hardhat assembly further includes a second earplug device; and an elongated flexible second tether having a distal portion thereof mounted to the second earplug device. The tether support structure further includes a second reel mechanism having a second spool rotating about a second spool axis. The second tether includes a proximal portion that cooperates with the second spool for rotation about the second spool axis between a respective first position and a respective second position. In the first position of the second tether proximal portion, the elongated second tether retractably and rotatably cooperates with second spool such that the distal portion of the flexible first tether and the first earplug device are positioned proximate to the protective housing, in a retracted condition, In the second position, the distal portion of the flexible first tether and the first earplug device can be repositioned away from the protective housing by a selected distance, enabling usable access to the second earplug device, in an extended condition.

In yet another specific arrangement, the protective housing includes a first housing portion, supporting the first reel mechanism, and a second housing portion, supporting the second reel mechanism thereon. The first housing portion and the second housing portion are configured to cooperatively articulate relative to one another.

### BRIEF DESCRIPTION OF THE DRAWINGS

The assembly of the present invention has other objects and features of advantage which will be more readily apparent from the following description of the best mode of carrying out the invention and the appended claims, when taken in conjunction with the accompanying drawings, in which:

FIG. 1 is a rear perspective view of a hardhat incorporating a retrofit earplug assembly with a retractable tether apparatus and an earplug device constructed in accordance with the present invention, illustrated in a retracted position.

FIG. 2 is a rear perspective view of the hardhat and retrofit earplug assembly of FIG. 1, in an extended position.

FIG. 3 is a bottom perspective view of the hardhat and retrofit earplug assembly of FIG. 1, in the retracted position.

FIG. 4 is a bottom perspective view of the hardhat and retrofit earplug assembly of FIG. 1, in the extended position.

FIG. 5 is an enlarged, side perspective view of the retrofit earplug assembly of FIG. 1, removed from the hardhat, and illustrating one embodiment with a Velcro backing.

FIG. 6 is a top plan view of the earplug assembly of FIG. 5, in the retracted position.

FIG. 7 is a top plan view of the earplug assembly of FIG. 5, in the extended position.

FIG. 8 is an enlarged bottom perspective view of the earplug assembly of FIG. 1, mounted to the hardhat, in the extended position.

FIG. 9 is a side elevation view, in cross-section, of the guide assembly of the earplug assembly of FIG. 5.

FIG. 10 is a bottom perspective view of an alternative embodiment of the retrofit earplug assembly of FIG. 1, having dual earplug devices shown in the extended position.

FIG. 11 is a side perspective view of an alternative embodiment retrofit earplug assembly of FIG. 1, in an extended position.

FIG. 12 is a side perspective view of the alternative embodiment retrofit earplug assembly of FIG. 11, in a retracted position.

FIG. 13 is a bottom perspective view of another alternative embodiment of the retrofit earplug assembly of FIG. 1, mounted to a hardhat and in a retracted position.

FIG. 14 is a bottom perspective view of a modified hardhat assembly that incorporates a track slots formed and dimensioned for receipt of the earplug assembly of FIG. 13.

FIG. 15 is a side perspective view of a protective housing device of the earplug assembly of FIG. 13.

FIG. 16 is a bottom perspective view of still another alternative embodiment of the retrofit earplug assembly of FIG. 1, removably mounted to a brim portion of the hardhat and in a retracted position.

FIG. 17 is a top perspective view of the retrofit earplug assembly of FIG. 16, illustrating mounting to the hardhat brim portion.

FIG. 18 is a top perspective view of the protective housing of the earplug assembly of FIG. 16.

FIG. 19 is a side elevation view of the earplug assembly of FIG. 16, in the retracted condition.

FIG. 20 is a side elevation view of the earplug assembly of FIG. 16, in the extended condition.

FIG. 21 is a bottom perspective view of the earplug assembly of FIG. 16.

FIG. 22 is a bottom perspective view of yet another alternative embodiment of the earplug assembly of FIG. 1 having a track assembly molded into the hardhat.

FIG. 23 is a top perspective view of still another alternative embodiment of the earplug assembly of FIG. 1, incorporating a retractable reel mechanism.

FIG. 24 is a bottom perspective view of the earplug assembly of FIG. 23, shown in the retracted condition and mounted to the brim portion of the hardhat.

FIG. 25 is a bottom perspective view of the earplug assembly of FIG. 24, shown in the extended condition.

FIG. 26 is a side elevation view of the earplug assembly of FIG. 24, illustrating the reel mechanism.

FIG. 27 is a top perspective view of the earplug assembly of FIG. 23, shown in the retracted condition and mounted to an exterior wall of the hardhat crown portion.

FIG. 28 is a bottom perspective view of the earplug assembly of FIG. 23, shown in the retracted condition and mounted to an interior wall of the hardhat crown portion.

FIG. 29 is a top plan view of an alternative embodiment of the earplug assembly of FIG. 23, incorporating a single earplug device.

FIG. 30 is a top plan view of an alternative embodiment of the earplug assembly of FIG. 23, incorporating dual earplug devices.

FIG. 31 is a bottom perspective view of the earplug assembly of FIG. 30, in the extended condition.

FIG. 32 is an exploded top perspective view of a retractable reel mechanism for the earplug assemblies of FIGS. 23-31.

FIG. 33 is an exploded top perspective view of an outer enclosure device for the earplug assemblies of FIGS. 23-31.

FIG. 34 is a top perspective of still another alternative embodiment earplug assembly of FIG. 23.

### DETAILED DESCRIPTION OF THE INVENTION

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various figures.

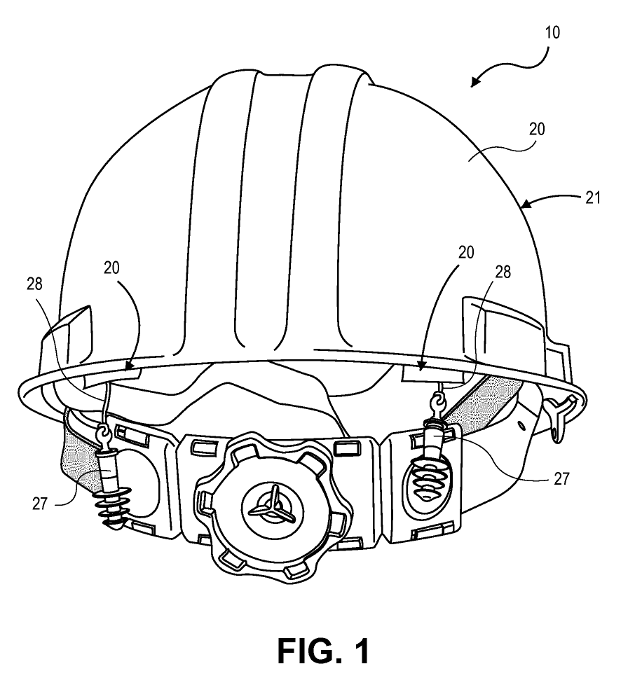

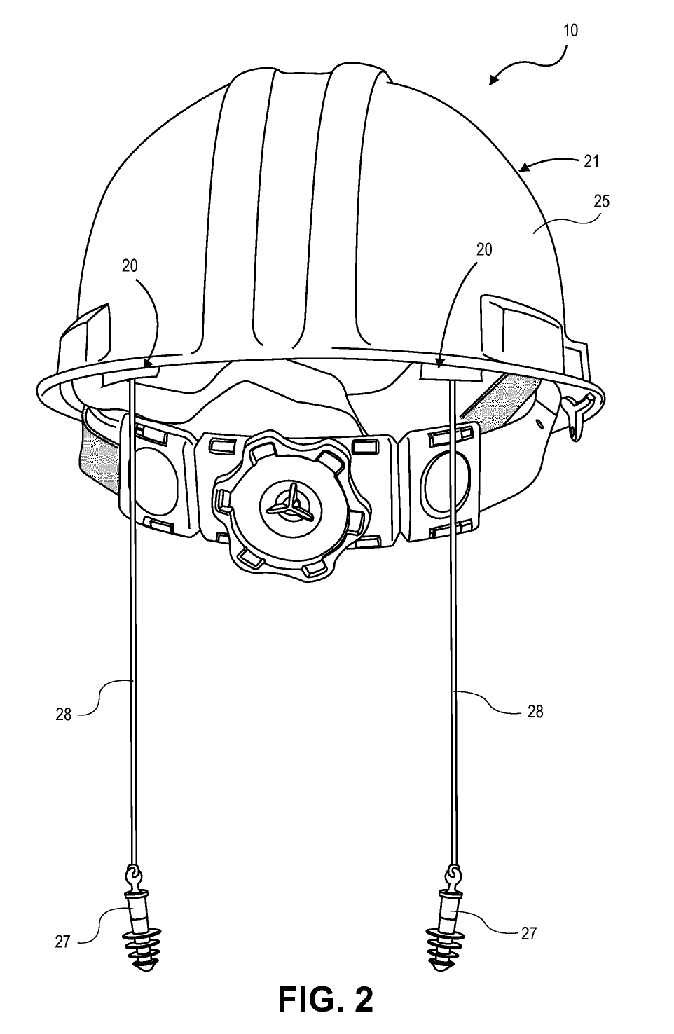

Referring now to the embodiment of FIGS. 1-7, although the initial description applies to nearly all embodiments, a hardhat assembly 10 is provided including a headwear device 21, preferably a hardhat, and a retractable earplug assembly 20 removably mounted to the hardhat. This earplug assembly 20 includes a tether support structure 12 and a protective housing 24 that defines a mount support portion 11. A mounting assembly 22 cooperates with the support portion 11 and a surface of the hardhat 21 to removably mount the protective housing thereto. The housing 24 supports an elongated flexible tether 28 having a distal portion thereof mounted to the earplug device 27, and a proximal portion thereof movably mounted to the housing between a first position (FIGS. 1, 3, 5 and 6) and a second position (FIGS. 2, 4 and 7). In the first position of the tether proximal portion, the elongated tether retractably cooperates with the tether support structure 12 such that the distal portion of the flexible tether and the earplug device are positioned proximate to the protective housing, in a retracted condition (FIGS. 1, 3, 5 and 6). In contrast, in the second position of the tether proximal portion, the a distal portion of the flexible tether 28 and the earplug device 27 can be repositioned away from the protective housing 24 by a selected distance, enabling usable access to the earplug device 27, in an extended condition (FIGS. 2, 4 and 7).

Accordingly, a retractable earplug assembly is provided that can be retrofit mount to any headwear, but preferably a hardhat. When the tether and earplug devices are oriented in the extended condition, the extended tether is of a sufficient length that enables the user to access the earplugs, and place them in the user's ear for use when the headwear is being worn. When the tether and earplug devices are oriented in the retracted condition, the tether 28 retracted into or by the tether support structure 12 in a manner stowing away the tether and the position the earplugs close to the housing during non-use.

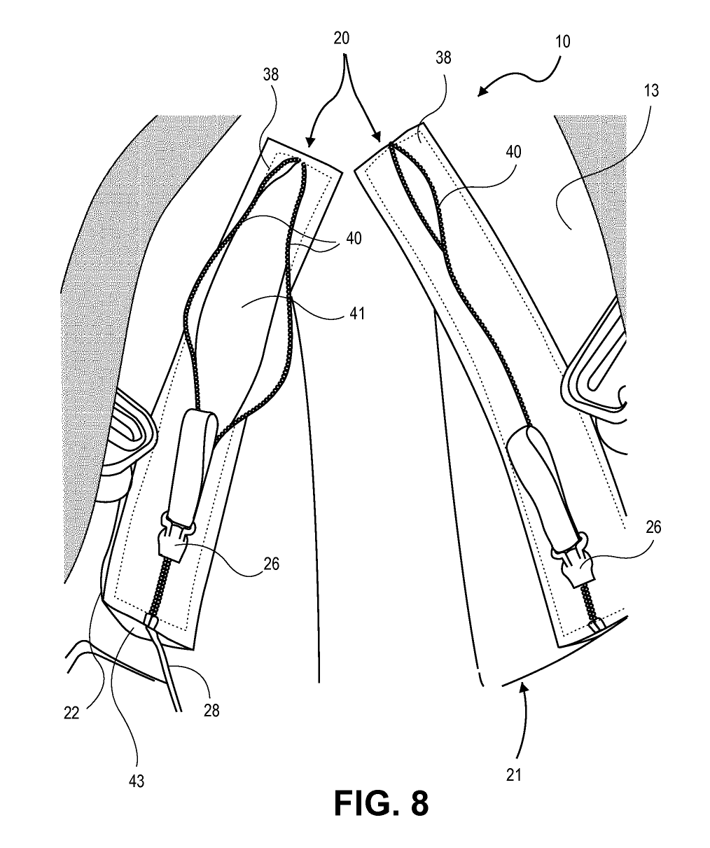

In one specific embodiment, as shown in FIGS. 1-8, the earplug assembly includes an elongated protective housing 24. The tether support structure 12 defines an interior channel 41 extending generally from one end of the housing to an opposite end 14 thereof, and further defines an elongated guide portion 23, extending substantially adjacent the interior channel 41. In one particular embodiment, the housing 24 is removably mounted to an interior wall 13 of the hardhat 21 through a mounting assembly 22 which is configured to cooperate with the mount support portion 11 and the interior wall of the crown portion 25 for removable mounting therebetween.

The earplug assembly 20 further includes a guide base 26 configured to slideably cooperate with the guide portion 23 for movement thereof along a fixed path in a direction generally longitudinally along and substantially adjacent the interior channel between the first position (FIGS. 1, 3, 5 and 6) and the second position (FIGS. 2, 4 and 7). A distal portion of the elongated flexible tether 28 is mounted to the corresponding earplug device 27, and a proximal portion thereof is mounted to the guide base 26. Preferably the distal portion of the elongated tether is at the distal end thereof. Similarly, the proximal portion of the elongated tether is preferably at the proximate end thereof.

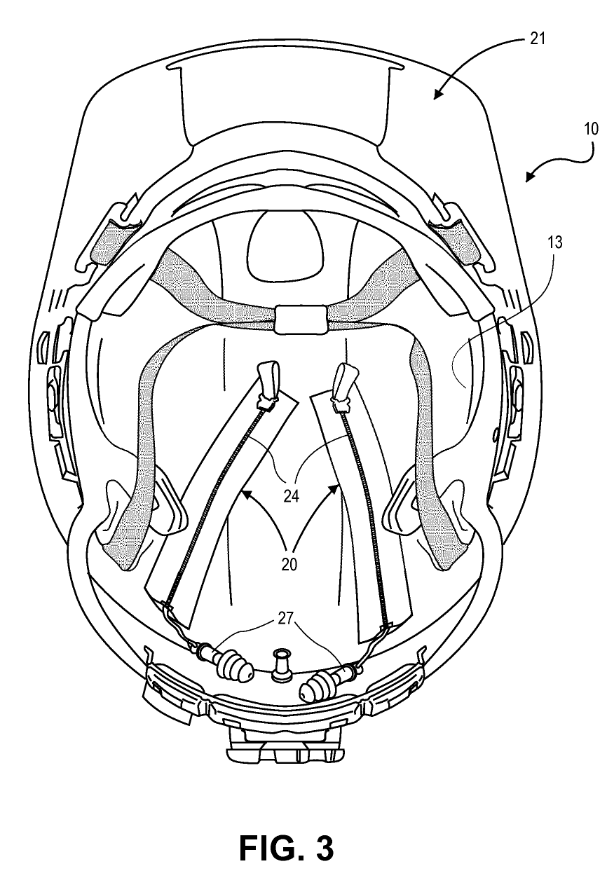

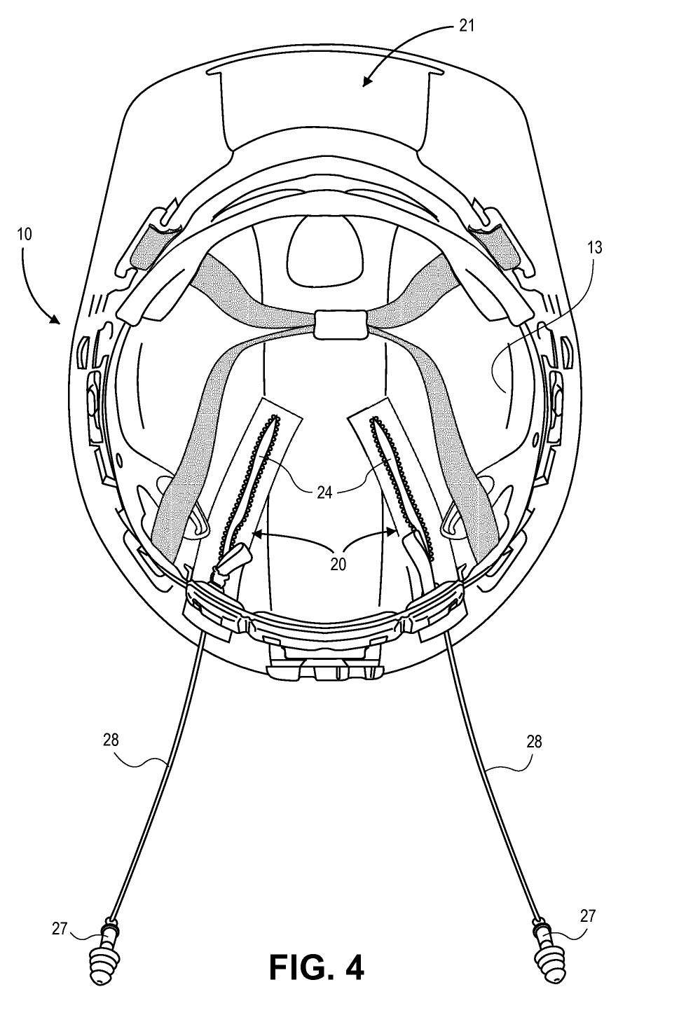

When the guide base 26 moves along the fixed path between the first position (FIGS. 3 and 6) and the second position (FIGS. 4 and 7), the tether 28 and the earplug device 27 move between a retracted condition (FIGS. 1, 3 and 6) and an extended condition (FIGS. 2, 4 and 7). As mentioned, in the extended condition, when the hat is being worn, the flexible tether 28 is extended to allow operational access to the earplug devices 27.

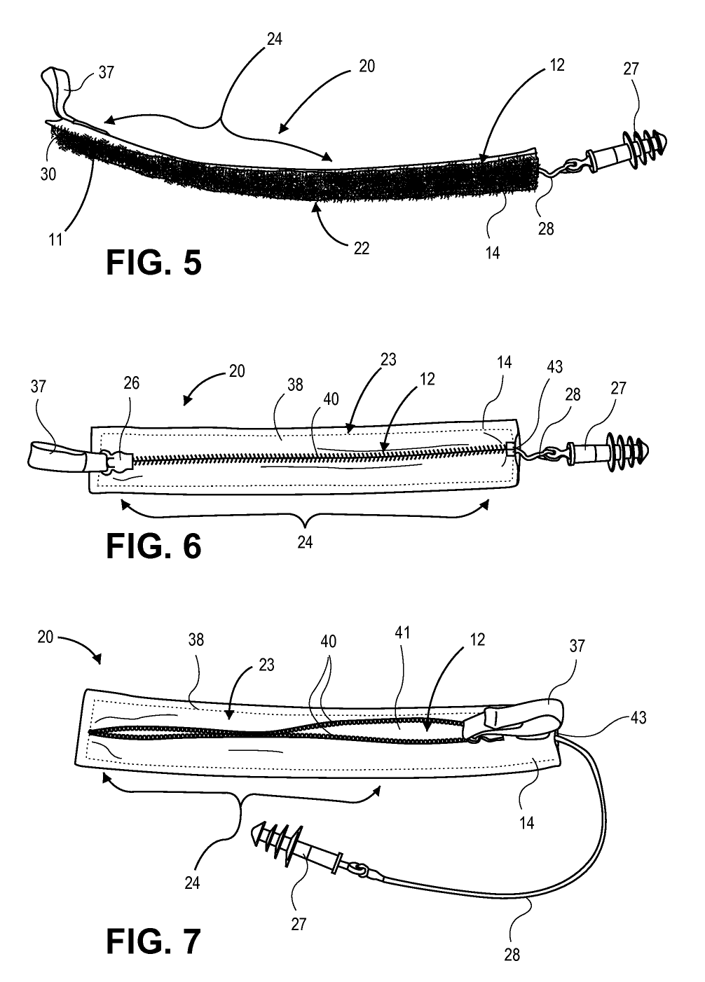

Referring now to FIGS. 5-7 and 9, in this particular configuration, each earplug assembly 20 includes a protective housing composed of a flexible material. Such flexibility enables the housing to conform to the curvature of the hardhat interior wall 13 upon removable mounting thereto. The mount support portion 11 includes a flexible material backing 30 that cooperates with the mounting assembly 22, (e.g., an adhesive or VELCRO®-type fastener) for removable affixation to the hardhat 21, preferably along the underside interior wall of the crown portion 25 thereof. Such a mounting assembly may comprise any type of adhesive, VELCRO®-type fastener, buttons, press-fit snaps, etc. that enable removable securing of the earplug assembly to the housing to the hardhat interior wall. Preferably, a VELCRO®-type fastener (e.g., hook and loop material) may be sewn or adhered to the material backing 30 of the housing and to the interior wall 13 of the hardhat. Such a fastener is preferable due in part to its ease of operation and placement, and its removable fastening, yet secure mounting.

A topside material backing of the housing 24, in this embodiment, incorporates the guide portion 23 and the guide base 26 that is movably mounted to the guide portion 23 for movement thereof along a fixed path between the first position (FIGS. 1, 3 and 6) and the second position (FIGS. 2, 4 and 7). As mentioned, the flexible tether 28 is provided having a distal portion mounted to the earplug device 27, while a proximal portion of the tether is mounted to the guide portion. The retrofit earplug assembly 20 is structured such that when the guide base 26 moves along the fixed path between the first position and the second position, the distal portion of the tether and the earplug device also correspondingly move between the extended condition (FIGS. 1, 3 and 6) and a retracted condition (FIGS. 1, 3 and 6), respectively, out of or into an opening 43 at one end of the housing 24.

In the retracted condition, the guide base 26 and the mounted tether 28 cooperate to position and slideably move the tether along the fixed path wherein the earplug device is drawn in and positioned adjacent or proximate to the opposite end 14 of the housing 24. Accordingly, by orienting the placement of the opposite end 14 of the housing 24 proximate to the bottom edge portion 15 of the crown portion 25, the earplug devices 27 can also be oriented proximate thereto when in the retracted condition. As shown in FIGS. 1, 3 and 6, in this position, the tether does not freely hang or dangle about the hat to prevent entanglement. A much more organized and cleaner appearance is provided. Moreover, this position enables easy access to grip the earplug so that the user can manually pull the tether and earplug device toward the extended condition. In the extended condition, of course, the tether is extended from the hardhat to enable the earplug device 27 to be placed in the user's ears for use.

A pull tab 37 is included that is mounted to the guide base 26 to facilitate manual manipulation thereof between the first position and the second position. Moreover, as best illustrated in FIGS. 5, 6 and 9, the proximal portion of the tether 28 is mounted to the guide base 26, as well. Accordingly, as the guide base 26 is manually moved or pulled along the guide portion 23 between the retracted and extended positions, the flexible tether 28 is drawn along the fixed path which in turn moves the earplug device between the extended (FIGS. 2, 4 and 7) and retracted conditions (FIGS. 1, 3 and 6), respectively.

The tether 28 is preferably composed of a relatively flexible non-elastic material that provides sufficient tensile strength to secure the earplug device 27 thereto. Such tether flexibility is required to permit operational use of the earplug device 27 when the hat is being worn, and when the tether is oriented in the extended condition. Furthermore, the tether must be capable of conforming to the profile of the crown portion 25, along the fixed path, when the tether 28 is moved to the retracted condition. Many cord or strap materials can be satisfactorily employed such as rope, twine, plastic, nylon, and other fabrics. One specific example of the tether material is a tether ribbon composed of fabric. While a non-elastic tether material is preferred, an elastic material can be employed, however.

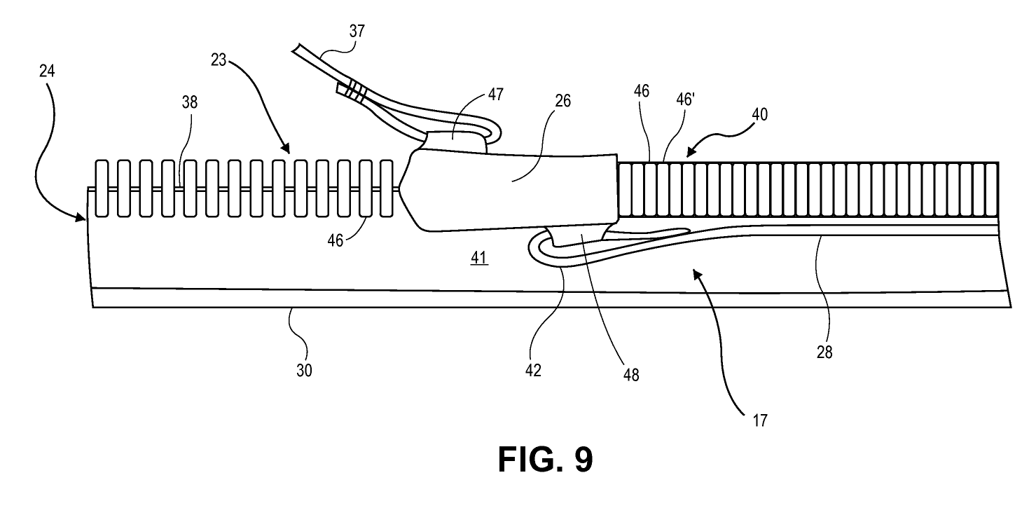

Turning now to FIG. 9, the guide portion 23 includes a topside material backing 38 that supports a guide track 40 upon which the guide base 26 is movably mounted. The topside material backing 38 is comprised of an elongated, rectangular material having sufficient flexibility to conform to the curvature or profile of the crown portion 25 of the hat. The guide track 40, similarly is composed of a material sufficiently flexible to enable conformance of the guide track to the curvature or profile of the crown portion 25, yet provide sufficient rigidity and support to enable the guide base 26 to move, slide and/or track therealong. Such materials include virtually any type of fabric.

To accommodate and retain the flexible tether along the fixed path, the elongated support portion 11 of the protective housing 24 further includes a bottom side material backing 30 that cooperates with the topside material backing 30 to form an elongated interior channel 41 extending substantially along the fixed path between the first position and the second position. This interior channel 41, as best viewed in FIGS. 7-9, is sized and dimensioned to slideably receive into and deliver therefrom the tether 28, mounted at the proximal portion 42 to the guide base 26, as the guide base reciprocates along the guide track 40.

The elongated interior channel 41 is created along the fixed path by affixing the opposed longitudinal edge portions of the rectangular topside material backing 38 to the bottom side material backing 30. As indicated, the opposed edges of the guide portion are affixed to the corresponding edges of material backing 30. At one end of the interior channel is the opening 43 that is formed to receive into and deliver therefrom the tether as the guide base moved along the guide track 40.

As the guide base 26 is manually moved, via pull tab 37, along fixed path of the guide track from the second position, where the tether 28 is in the corresponding retracted condition (FIGS. 1, 3 and 6), to the first position, where the tether is in the corresponding extended condition (FIGS. 2, 4 and 7), the tether is therefore delivered from storage in the interior channel 41 through the interior channel opening 43. Another technique to deliver the tether is by simply pulling on the earplug device 27, which in turn draws the tether through the interior channel opening 43 to the retracted condition, and displaces the guide base 26 from the first position to the second position. This provides a sufficient extension or length of tether ribbon to enable mounting of the earplug device 27 to the user's ear.

During periods of non-use, the tether 28 can be retracted back into the interior channel 41 through the interior channel opening 43. This is performed by manually manipulating the guide base 26, via pull tab 37. By pulling the guide base along the guide track from the first position (FIGS. 2, 4 and 7) to the second position (FIGS. 1, 3 and 6), the tether 28 which is attached to the guide base 26 at the one end, is pulled and retracted back through the interior channel opening 43 and along the fixed path of the interior channel 41 toward the retracted condition.

It will be understood that the extension length of the tether 28 can be controlled by the length of the fixed path for the first position to the second position. For example, should the guide portion 23 only extend from the top of the crown portion 25 to the rear bay portion 45 of the hardhat 21, the extension length of the tether 28 from the retracted condition to the extended condition will be proportionately shortened. Thus, depending upon the application, the tether extension can be adjusted accordingly.

In accordance with the present invention, as the head or guide base 26 moves, slides and/or ratchets along the guide portion 23, the tether is retained along the fixed path, substantially eliminating tether dangle. The guide portion, in one specific embodiment, is provided by a rail-type structure (i.e., the guide track 40) that cooperates with the guide base 26 to move both the guide base and the one end of the tether along the fixed path. In one example, a ratchet type structure maybe provided that locks the guide base along the fixed path between the first position and the second position unless the guide base, and hence the tether, is released. Other sliding-type devices suitable for use along the guided fixed path include plastic zippers and enclosed glide and pull devices.

More specifically, the example illustrated in FIGS. 5-7 and 9 represent a conventional zipper style mechanism. The guide track 40, thus, is provided by the zipper teeth or chain portion of a zipper, while the guide base 26 is provided by a zipper slider. Thus, when the zipper slider slides or tracks along the zipper teeth 46, 46′ between the first position (FIGS. 2, 4 and 7) and the second position (FIGS. 1, 3 and 6), the tether ribbon 28 is moved between the extended condition and the retracted condition, respectively.

The pull tab 37 or the like is included mounted to a lower bracket 47 of the zipper slider for manual manipulation of the zipper between the first position and the second position. Similarly, the proximal portion 42 of the tether ribbon 28 is mounted to an upper bracket 48 of the zipper slider 26.

In this particular sample, when the zipper slider 26 is moved to the first position, retracting the tether into the interior channel 41, the zipper mechanism is zipped or closed. In contrast, when the zipper slider 26 is moved to the second position, delivering the tether from the interior channel 41, the zipper mechanism is unzipped or opened.

In one configuration of this embodiment, the protective housing contains one tether 28 and one earplug device 27. Accordingly, as shown in FIGS. 1-4 and 8, two independent earplug assemblies 20, 20′ are required, one for each ear. Although the positioning of the earplug assemblies is such that the earplug devices 27 are positioned proximate to the lower edge portion 15 of the hardhat when the protective housing is mounted to the hardhat and when the tether and earplug device are oriented in the retracted condition, it will be appreciated that earplug assemblies can be removably mounted essentially anywhere along the hardhat.

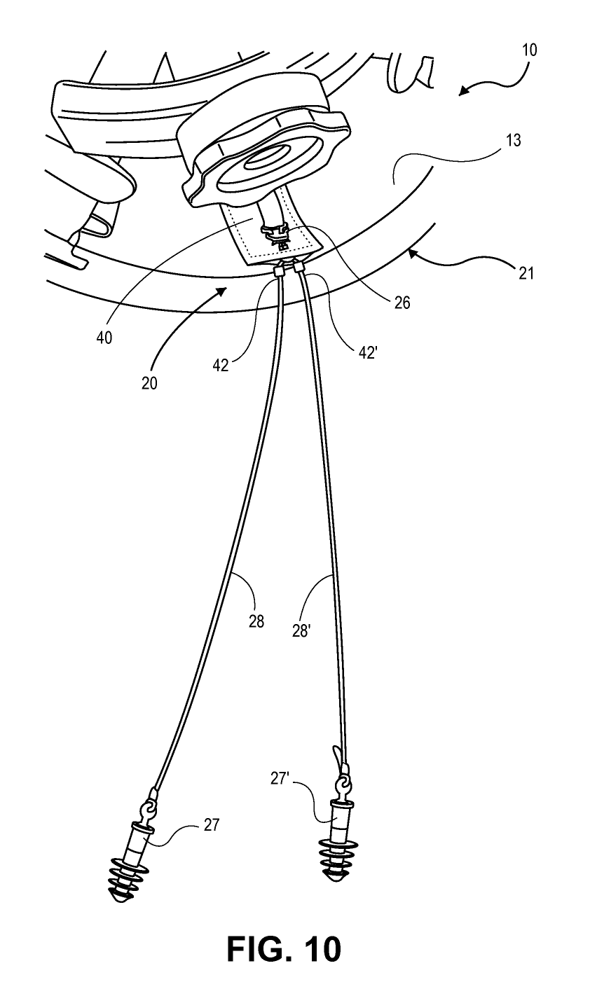

In another configuration of this embodiment, two tethers 28, 28′ and two corresponding earplug devices 27, 27′ are mounted to a single guide base 26 of a single protective housing 24. As best illustrated in FIG. 10, the proximal portions 42 of each tether 28, 28′ are mounted to guide base 26. Accordingly, as the guide base 26 moves along the guide track 40 from the second position (FIG. 10) to the first position (not shown), the tethers 28, 28′ simultaneously move from the extended condition (FIG. 10) to the retracted condition (now shown) where both tethers are to be contained in the interior channel 41 thereon.

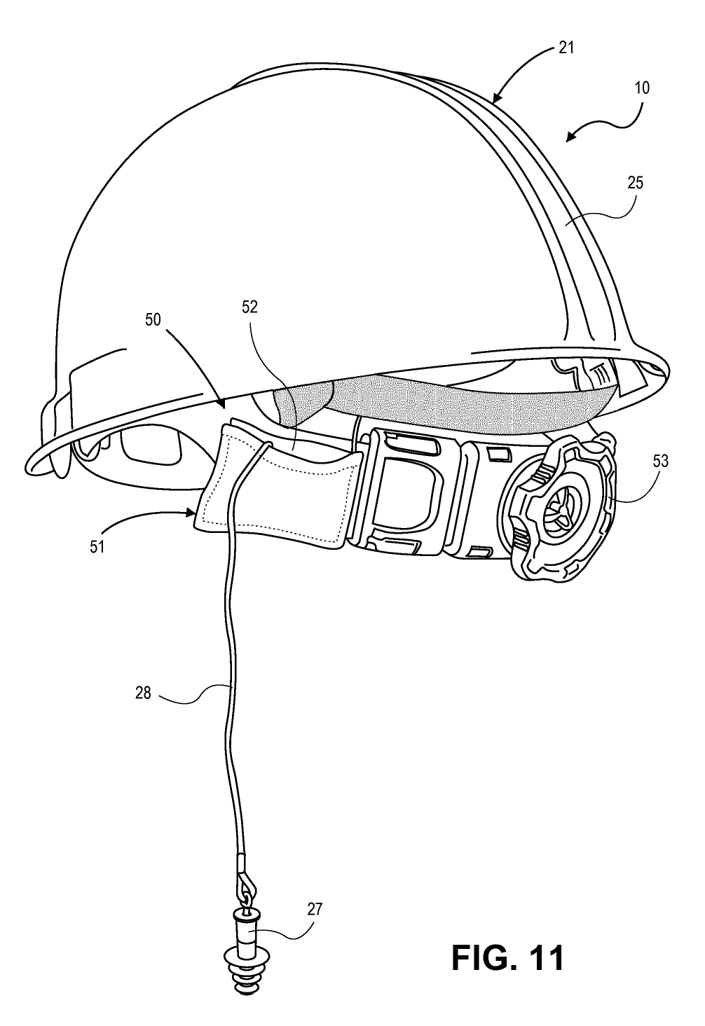

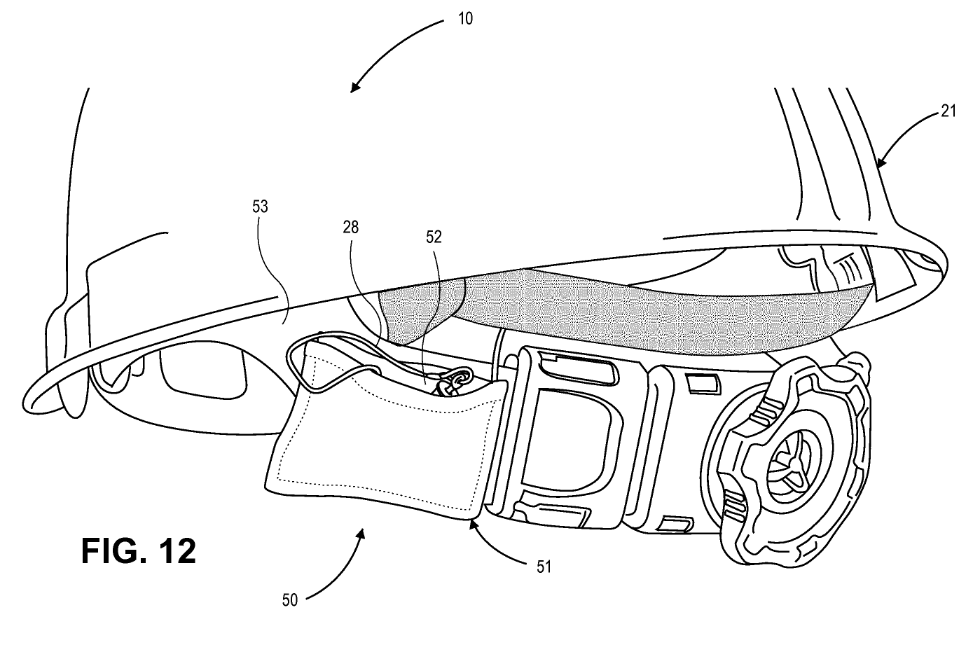

Referring now to FIGS. 11 and 12, an alternative embodiment retrofit earplug assembly 50 is provided that also includes an earplug device 27 mounting to an end of the tether 28. In this embodiment, the earplug assembly 50 includes a housing 51 that defines an interior pocket 52 sized for receipt of the tether 27 and earplug device 27 therein for storage and periods of non-use (FIG. 12). The opposite end of the tether 28 is mounted or affixed to the housing 51 inside the interior pocket thereof. Accordingly, during use, the tethered earplug device 27 can be easily accessed and removed from the interior pocket 52 (in the extended position shown in FIG. 11).

In one particular embodiment, the housing 51 can be mounted to an adjustable head strap 53 or the like of the hardhat 21, using VELCRO® or any other common mounting device or adhesive. It will be appreciated that the housing 51 of the retrofit earplug assembly 50 may also be mounted to the exterior or interior of the crown portion 25 as well.

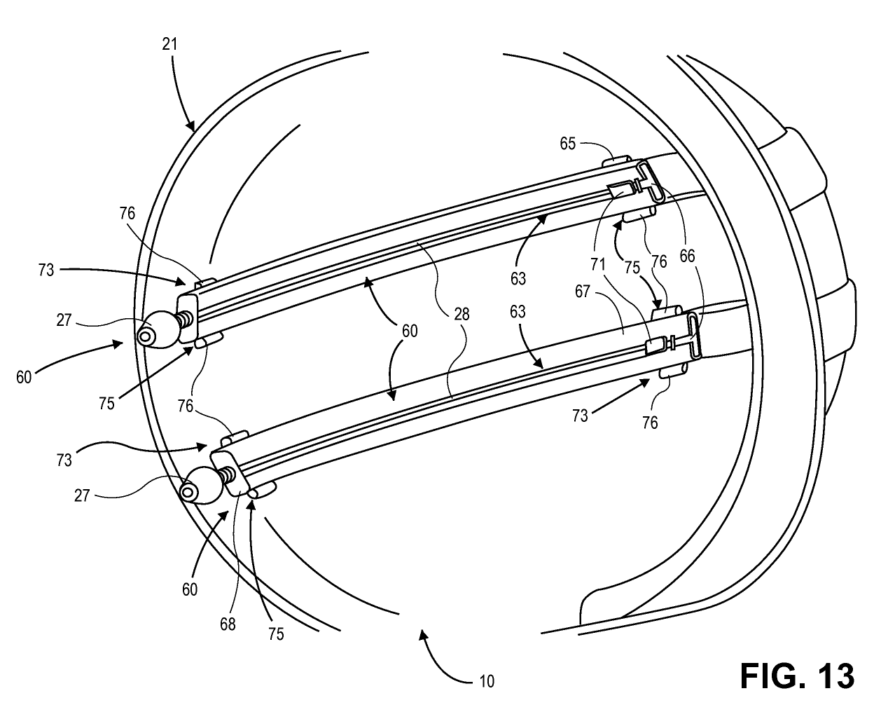

Turning now to FIGS. 13-15, a more rugged design of the retrofit earplug assembly 60 is illustrated. In one specific embodiment, the earplug assembly is designed to more or less removably hard mount into the designated OEM channels 61a, 61b of a hardhat 21 with only minor modifications by the hardhat manufacturers. Typically, hardhats 21 include these interior channels, typically spaced-apart pair that extends from the front side of the hardhat to the rear side thereof. By using these OEM channels, the earplug housings can be easily aligned and seated therein with only minimal modifications, as will be described, such as the adjusting the width and depth of the channels.

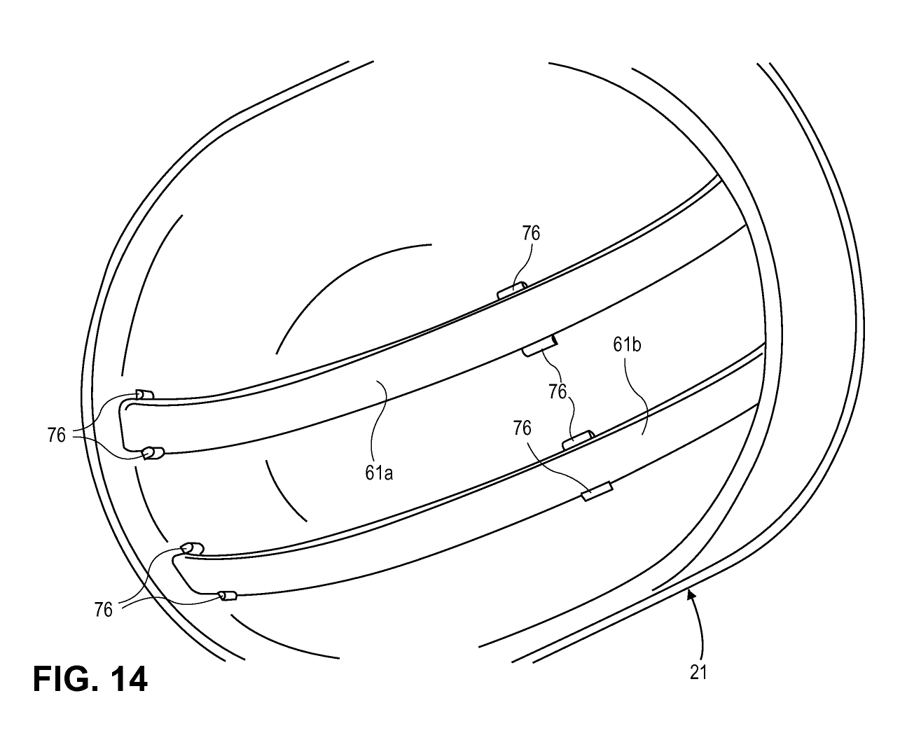

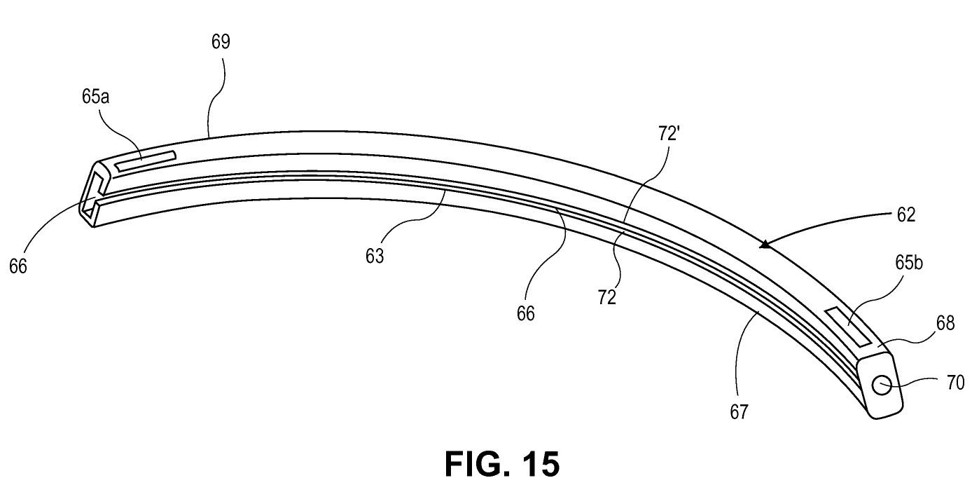

As best illustrated in FIGS. 13 and 15, these retrofit earplug assemblies 60 include a relatively non-deformable, elongated protective housing 62 that is preferably composed of a thermo-elastic polymer material such as plastic or the like. This is beneficial in that the housing is significantly more rugged, and yet easily moldable.

The protective housing 62 includes an upper guide portion 67, a tether support structure 63 and an opposed mount support portion 69 that is configured to have a curvature generally conforming to that of the rear portion interior wall of a corresponding channel 61a, 61b (FIG. 14). As will be described in greater detail below, such conformation enables seating and aligned support against the interior wall 13 of the hardhat 21.

The tether support structure 63 of each relatively rigid protective housing 62 defines an elongated channel 66 extending from one portion to an opposite portion 68. As shown in FIG. 20, this channel 66 has a generally T-shaped transverse cross sectional dimension. At the closed rear side distal end of the protective housing is an opening 70 that extends into the T-shaped channel 66 of the protective housing 62. This opening 70 is sized and dimensioned for reciprocal sliding receipt of the tether 28 therethrough.

The distal portion of the tether 28 is mounted to the corresponding earplug device 27, while the proximal portion thereof is mounted to a guide base 71 slideably seated in the T-shaped channel 66 for reciprocating movement therein. An upper portion of the guide base slideably engages the opposed flange portions 72, 72′ of the guide portion 67, while the lower portion thereof slideably engages the T-shaped channel. As mentioned above, the guide base 71 slides along the protective housing enabling the tether 28 and earplug device 27 to reciprocate from an extended position (not shown), enabling accessible operation of the earplug devices 27, and a retracted position (FIG. 13), storing and retracting the tether into the channel 66 of the protective housing 62.

Thus, similar to the previous embodiments, when the guide base 71 and the tether assembly 60 are in the retracted position, the earplug device 27 can be pulled manually toward the extended position, moving the guide base from a first position to a second position at the rear of the hardhat 21. Similarly, to move the tether back toward the retracted position, the guide base 71, via a latch, is manually manipulated slideably along the protective housing back from the second position toward the first position.

As mentioned above, the protective housing 62 is removably mounted to the interior wall of the hardhat, preferably in the OEM channels thereof. In accordance with this embodiment, the outer width of the mount support portion 11 generally conforms to the width of the hardhat channels 61a, 61b upon which the protective housing is inset. The tolerance between these components should be relatively small since the protective housing is press-fit or friction fit in the respective channel.

In one particular mounting configuration, the earplug assembly 60 includes a mounting assembly 73 having a plurality of spaced-apart tab devices 75 configured for friction fit mounting of the protective housing to the crown portion interior wall 13. In one arrangement, as shown in FIG. 14, the tab devices 75 are disposed in pairs at two or more spaced locations (e.g., a front set and a rear set) longitudinally along the channels. Each tab device 75 includes a tab members 76 up-standing from the interior wall 13 on opposite sides of each OEM channel 61a, 61b. Thus, the width spacing between each opposed each tab member 76 is sufficient to slideably receive the protective housing 62, in the width dimension, therebetween.

Each tab member 76 includes a respective nub portion that extends slightly transversely across the width of the channel. Corresponding to the size, position and dimension of the tab member nub portions of the mounting assembly 62 (when the housing is properly aligned in the corresponding OEM channel 61a, 61b are opposed front and rear slots or indents 65a, 65b formed in the protective housings 62. These corresponding mount components allow the earplug assemblies to be easily press-fit into the corresponding channel 61a, 61b until the nub portions of the tab members 76 are seated in the therein. Removal of these assemblies from the hardhat, on the other hand, is just as simple. It will be appreciated, however, that these assemblies can be mounted to the hardhat using any conventional mounting technique.

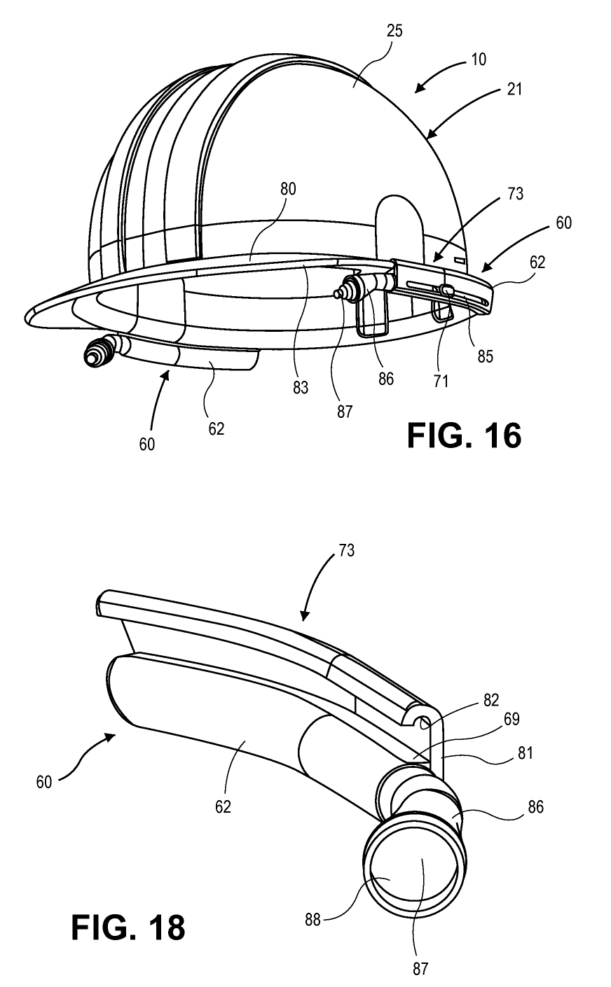

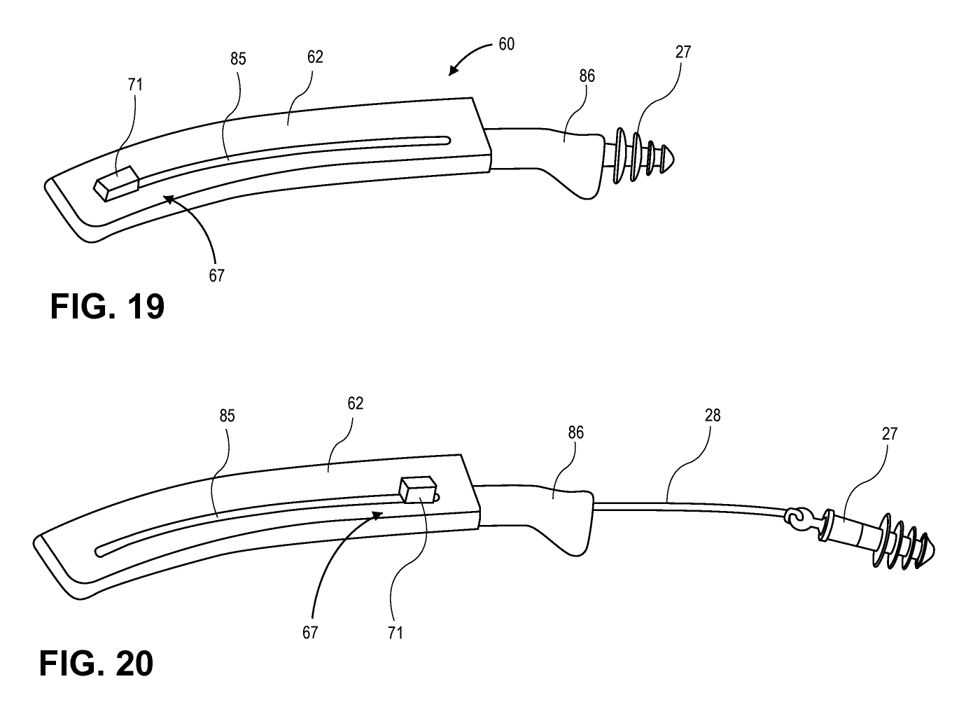

Turning now to FIGS. 16-21, an alternative embodiment earplug assembly 60 is illustrated having an elongated polymer protective housing 62 similar to the embodiments shown in FIGS. 13-15. In this configuration, however, the mounting assembly 73 is adapted to removably mount the housing to a brim portion 80 of the hardhat 21. In particular, the mounting assembly 73 of this earplug assembly embodiment is configured to removably affix the housing to a lip portion 83 at the peripheral edge of the hardhat brim portion 80.

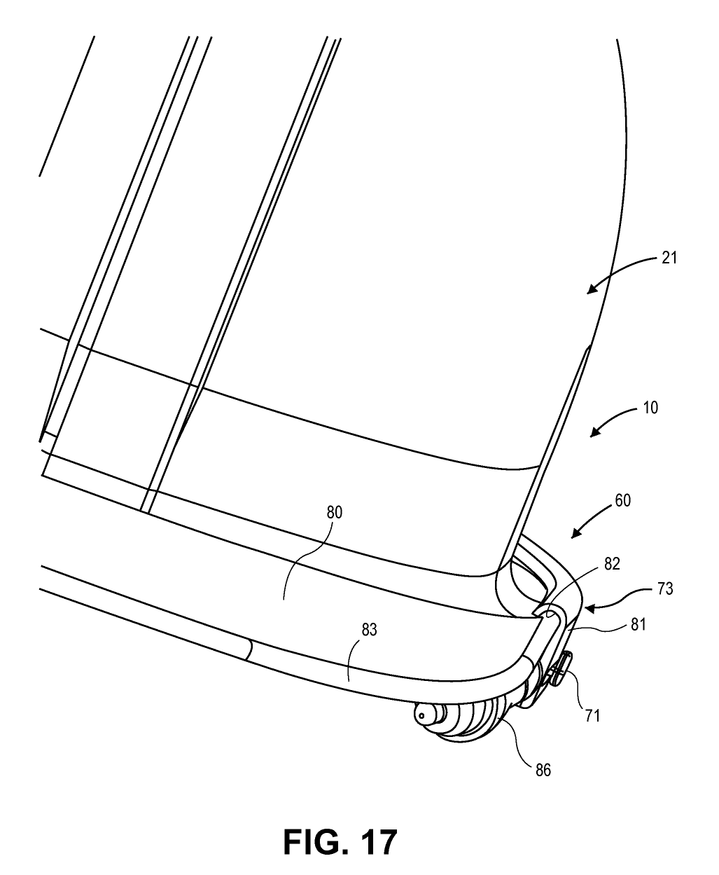

To mount the relatively rigid protective housing 62 to the brim portion 80, the mounting assembly 73 includes a tab portion 81 that defines a brim receiving socket 82 formed and dimensioned for cooperative receipt of the brim lip portion 83 therein. Moreover, the receiving socket 82 of the tab portion 81 is configured for friction-fit or press-fit receipt of the brim lip portion for securing mounting thereto. The receiving socket 82 therefore has a circular transverse cross sectional dimension (FIGS. 17 and 18), and should have a longitudinal curvature generally conforming to peripheral curvature of the region of the brim lip portion 83 selected for mounting thereto. Such conformity of course assists secured receipt of the brim lip portion 83 therein.

Although the mounting assembly 73, for this embodiment, has only been described as being a press-fit of friction-fit mounting, it will be appreciated that a fastener or an adhesive may be applied to facilitate mounting thereto. Further, although the tab portion 81 of the mounting assembly is illustrated as a single continuous unit upstanding from a mount support portion 69 of the protective housing 62, the tab portion can be provided by two or more tab members (not shown) spaced-apart longitudinally along the protective housing 62.

Lastly, the mounting assembly 73, in this specific embodiment, orients the protective housing just below the brim portion 80 of the hardhat 21. It will be appreciated, however, that the mounting assembly 73 can be easily configured to position the housing above and/or atop the brim portion 80, or generally vertically level therewith without departing from the true spirit and nature of the present invention.

As best illustrated in FIGS. 17, 18 and 21, the protective housing 62 is similarly of a curvature generally conforming to that of the peripheral edge of the brim portion. Accordingly, the corresponding tether support structure 63 also defines an interior channel 66 also having a slight curvature as well.

A guide portion 67 of the protective housing 62 defines a guide slot 85, formed between opposed flange portions 72, 72′ of the guide portion 67, which communicates with and extends substantially adjacent the interior channel 66. Similar to previous embodiments, a guide base 71 slideably cooperates with the interior channel 66 and the guide slot 85, via sliding engagement with the opposed flange portions 72, 72′, for reciprocating movement therein along a between fixed path between a first position (FIGS. 16, 19 and 21) and a second position (FIG. 20)

Again similar to the previous embodiments, a flexible tether 28 is provided having a proximal portion mounted to the slideable guide base 71, and an opposite distal portion thereof mounted to an earplug device 27. Accordingly, in the first position of guide base and the tether proximal portion, the elongated tether retractably cooperates with the tether support structure 12 such that the distal portion of the flexible tether and the earplug device are positioned proximate to the protective housing, in a retracted condition (FIGS. 16, 19 and 21). In contrast, in the second position of the tether proximal portion, the a distal portion of the flexible tether 28 and the earplug device 27 can be repositioned away from the protective housing 24 by a selected distance, enabling usable access to the earplug device 27, in an extended condition (FIG. 20).

In accordance with the present invention, extending distally from a distal portion of the protective housing is a parking structure 86 formed to seat the earplug device 27 therein during non-use, when the assembly is in the retracted condition. The parking structure 86 defines a passage 87 therethrough that is co-aligned with the housing interior channel 66, and is formed for sliding passage of the tether 28 therethrough.

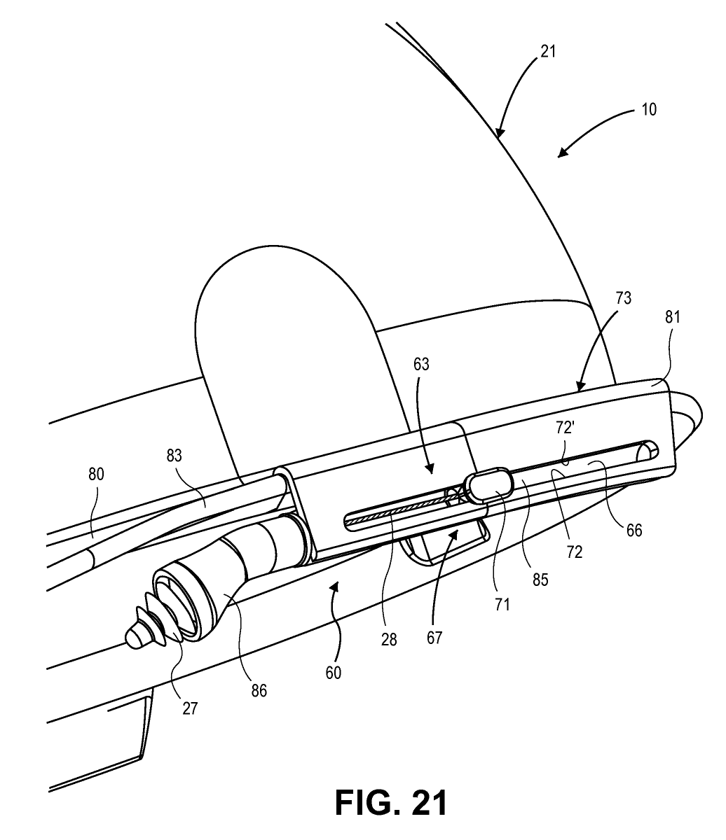

As best viewed in FIGS. 18 and 21, the parking structure includes a distal funnel portion 88 wherein the corresponding portion of the passage 87 funnels outwardly. The tapered shape and inner diameter of the funnel portion 88 is selected for seated receipt of at least a proximal portion of the earplug assembly therein, when the assembly is oriented in the retracted condition.

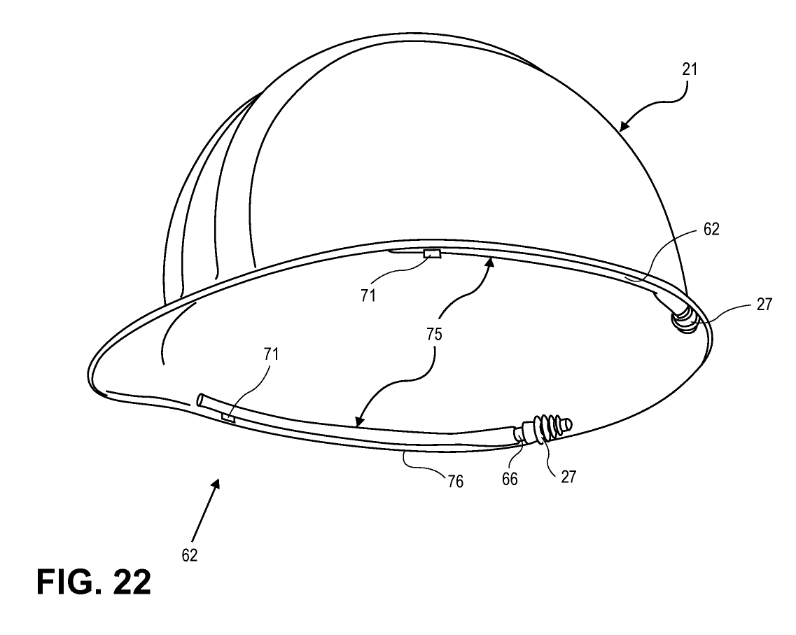

Referring now to FIG. 22, yet another alternative embodiment earplug assembly 75 device is shown. In this example, the protective housing 62 is integrally molded proximate to the lower edge portion 15 of the hardhat 21. The protective housing 62 defines a channel 66 formed and dimensioned for sliding reciprocal receipt of the tether therein. A sliding guide base 71 is formed to slide along the protective housing 62 between the first position (FIG. 22), containing the tether within the channel in a retracted condition, and a second position (not shown) at the other end of the protective housing, enabling extension of the tether and earplug devices to the extended position. Although not shown, a parking structure could be included to seat the earplug device, similar to the embodiments shown in FIGS. 16-21. Similarly, two tethers and corresponding earplug devices can be mounted to a single guide base (for the embodiments of FIGS. 16-22), as shown in the embodiment of FIG. 10.

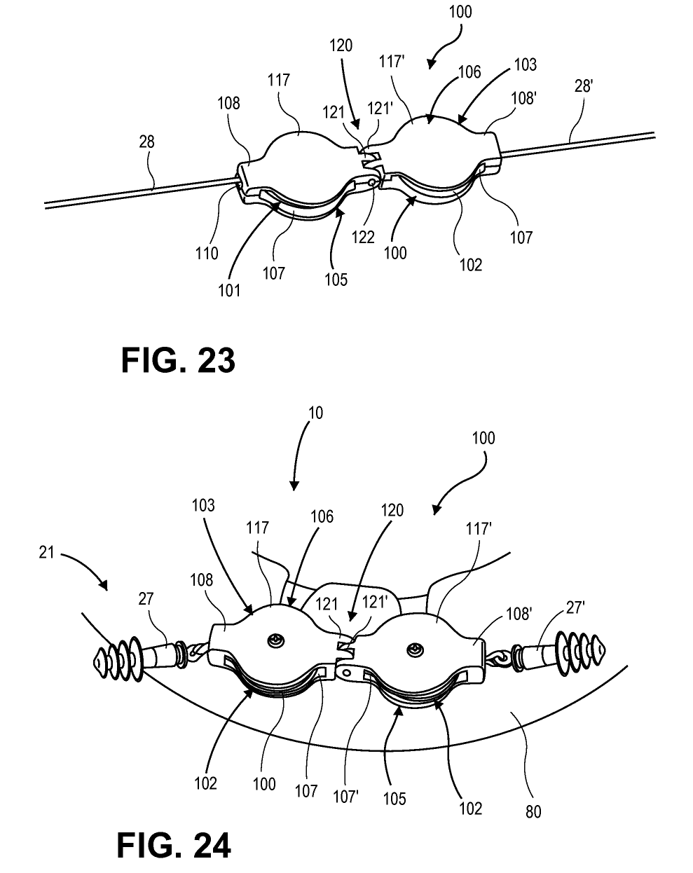

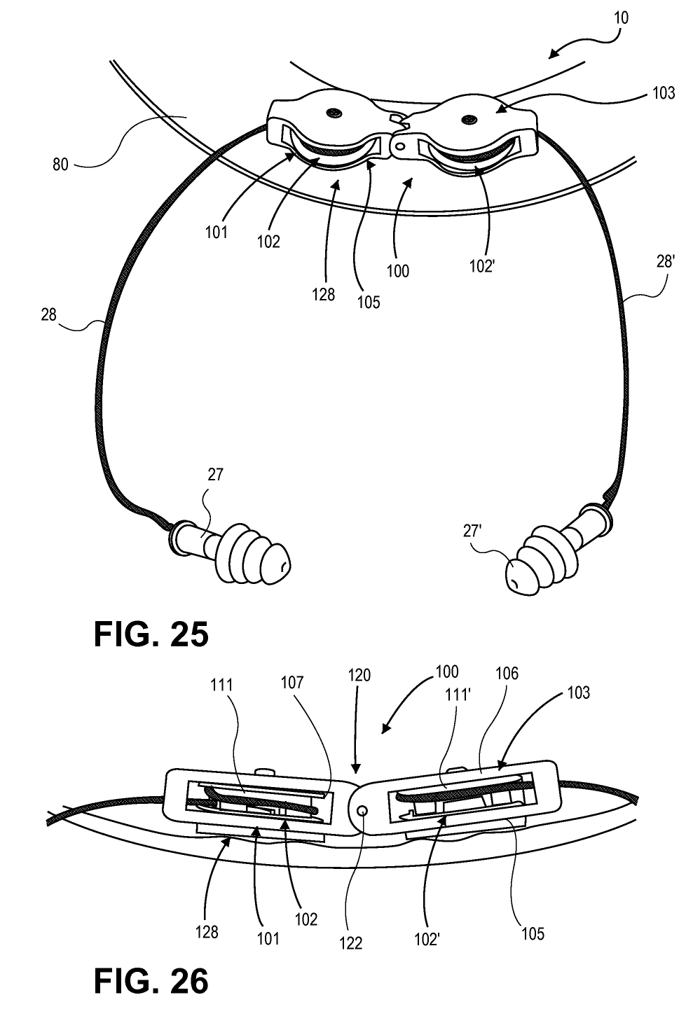

In another aspect of these retrofit earplug assemblies of the present invention, as best shown in FIGS. 23-31, a retrofit earplug assembly 100 is provided that includes a tether support structure 101 incorporating a retractable reel mechanism 102 formed for reeled movement of the tether 28 between a retracted condition (FIGS. 24, 27, 28 and 30) and an extended condition (FIGS. 25 and 31). Due in part to the spooled nature of the reel mechanism 102, a protective housing 103 of the earplug assembly 100 is provided that has a significantly smaller footprint than that of the elongated housing embodiments of FIGS. 1-22.

As shown in FIGS. 23, 29, 30 and 32, the protective housing 103 is generally disk-shaped, in all embodiments, having a lower mount support portion 105 and an opposed cover support portion 106. Collectively, the mount support portion 105 and the opposed cover support portion 106 cooperate to define an interior cavity 107 upon which the reel mechanism 102 of the tether support potion 101 is rotatably disposed for movement thereof between the first and second positions. The housing 103 further defines a spooling neck portion 108 having an opening 110 into the interior cavity 107. This opening 110 is formed for sliding receipt of the tether 28 as it reciprocates in and out of interior cavity between the retracted condition and the extended condition.

To retractably spool the tether mounted earplug device 27, a proximal portion of the tether 28 is mounted to a reel or spool 111 of the retractable reel mechanism 102. As shown in FIG. 32, which illustrates the retractable reel mechanism 102 in general, this spool 111, in turn, is freely rotatably disposed about an axial post member 112 upstanding into the interior cavity 107 from either the mount support portion (as shown) or the cover support portion 106.

The reel mechanism 102 further includes a coiled torsion spring 115 wound about the axial post member. An inner end of the coiled torsion spring is wedged in a receiving slot 113 of the axial post member 112 of the mount support portion 105, and an opposite outer end is mounted to the spool 111. Thus, by pulling the tether 28 and/or the earplug device 27 with a force sufficient to overcome the torsional force of the coiled torsion spring 115, the tether 28 can be extended through the opening 110, causing the mounted earplug device 27 to move from the retracted condition (FIGS. 24, 27, 28 and 30) to the extended condition (FIGS. 25 and 31).

The spool 111 is further caused to rotate around the axial post member 112 as the tether proximal end moves from the first position (FIGS. 24, 27, 28 and 30) to the second position (FIGS. 25 and 31). During rotation of the spool 111 about the axial post member 112, in a counter-clockwise direction in FIG. 32, the coiled torsion spring 115 is further tensioned, biasing the spool 111 and the proximal portion of the tether 28 back toward the first position from the second position. Consequently, the earplug device 27 is moved from the retracted condition to the extended condition, enabling usable access to the earplug device 27.

In one specific embodiment, upon manual release of the tether 28, the tensioned coiled torsion spring 115 causes the tether 28 to rewind about the spool 111 back toward the first position. Thus, the reel mechanism 102 will automatically rewind when the force applied to the tether 28 and/or earplug device 27 or is less than the torsional force of the coiled torsion spring 115.

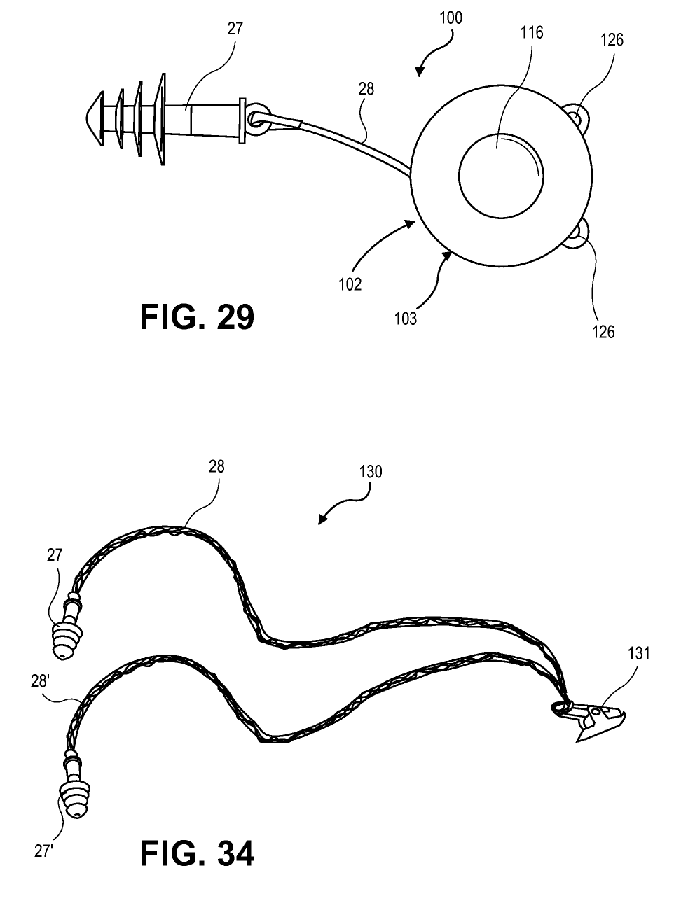

It will be appreciated that the reel mechanism 102 can be stepped or ratcheted, using conventional ratchet mechanisms (not shown) and techniques. The coiled torsion spring 115, thus, will be unable to rewind the spool 111 back toward the first position unless the ratchet mechanism is released. These ratchet mechanisms thus include a release device to enable manual release from the ratcheting. Conventional release mechanisms include those requiring full extension of the tether 28, for instance, before a release can commence and allow the spool to rewind the tether. Another example of a release mechanism is to provide an accessible release button 116, such as shown in the specific embodiment of FIG. 29. Operation of this manual release button 116 will release the ratchet mechanism, and enable the torsion spring to rotate the spool 111 and retract/rewind the tether 28 back to the retracted condition.

In accordance with this embodiment of the present invention, the earplug assembly 100 may incorporate single or dual earplug devices 27. For example, in the earplug assembly 100 of FIG. 29, a single earplug device 27 is provided that is mounted to single reel mechanism 102. In this embodiment, therefore, two independent earplug assemblies are required to be mounted to the hardhat.

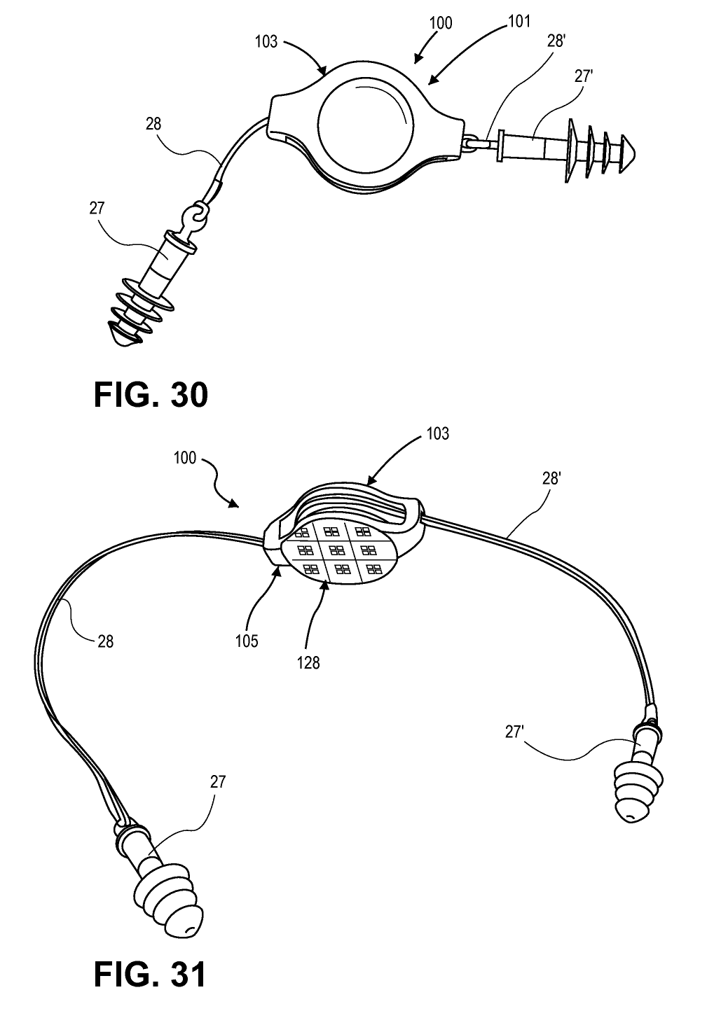

Referring now to FIGS. 30 and 31, an earplug assembly 100 is provided having dual earplug devices 27, 27′ mounted to a single reel mechanism 102. In this configuration, the earplug devices 27, 27′ are disposed on opposite sides of the spool 111 such that the respective tethers 28, 28′ are simultaneously wound and unwound from the spool during movement between the retracted and extended conditions. To facilitate simultaneous spooling of the tethers 28, 28′ from the housing 103, opposed spooling necks portion 108, 108′ are provided on opposed side thereof.

In still another configuration of the earplug assembly 100, as best illustrated in FIGS. 23-28, dual reel mechanisms 102, 102′ are provided, each supporting a respective tether 28, 28′ and corresponding earplug device 27, 27′ thereon. A protective housing 103 supports both reel mechanisms 102, 102′ therein, enabling independent operation thereof. Accordingly, the retraction and extension of each respective tether 28, 28′ and earplug device 27, 27′ combination can be operated independently of one another.

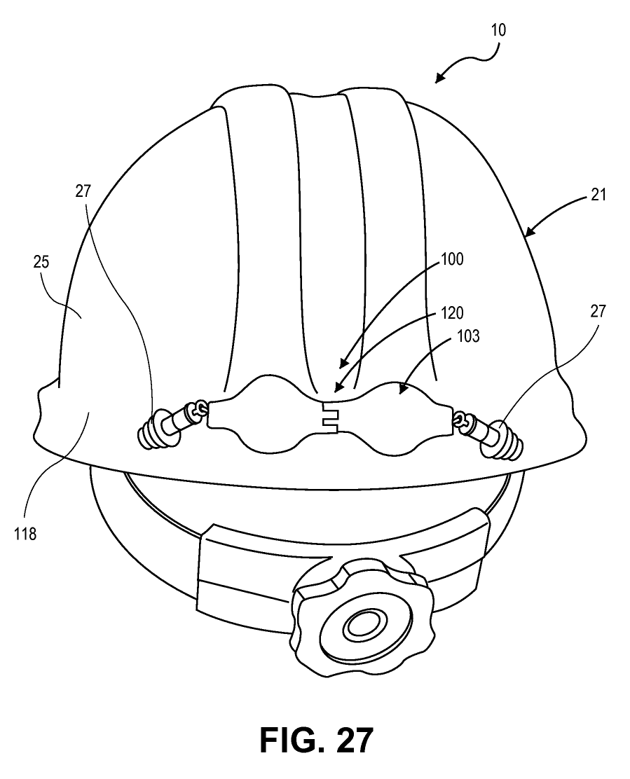

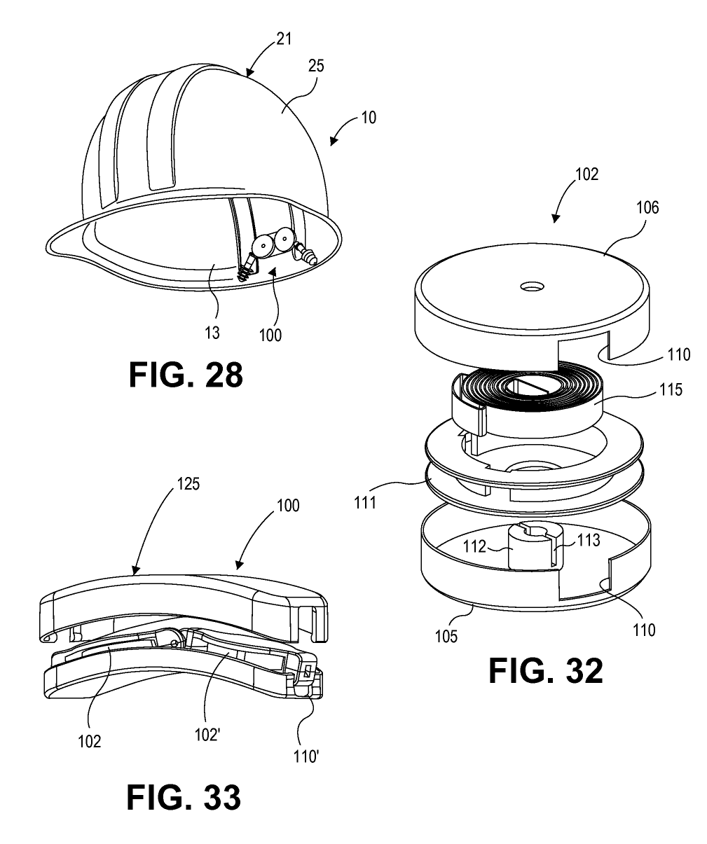

In one specific embodiment, the housing 103 may include a first housing portion 117 and a second housing portion 117′ mounted to one another in an articulating manner (FIGS. 23-28). Such articulation enables curved conformation of the respective mount support portions 105, 105′ to the selected mounting surface of the hardhat 21. The articulating protective housing 103, thus, can be mounted to a plurality of positions such as for example, to the interior wall 13 of the crown portion 25 (FIG. 28), the underside of the hardhat brim portion 80 (FIG. 25) or the exterior wall 118 of the crown portion 25 (FIG. 27).

The first and second housing portions 117, 117′ are hingedly mounted together through a hinge device 120. In one specific example, the hinge device 120 includes respective interlocking fingers or latches 121, 121′ both integral with their respective first and second housing portions 117, 117′. A hinge pin 122 extends through the nested interlocking fingers 121, 121′ to enable relative rotation about a hinge axis thereof.

In this particular configuration, each the first and second housing portion 117, 117′ defines a respective interior cavity, each formed for rotating support of the respective reel mechanism therein. Further, each housing portion 117, 117′ includes a spooling neck portion 108, 108′, disposed on opposite side the collective protective housing 103. The respective tethers 28, 28′ and earplug devices 27, 27′ are therefore delivered from the corresponding neck portions 108, 108′ in opposite directions thereof. It will be appreciated, however, that these neck portions 108, 108′ can be easily oriented at other locations circumferentially about the housing without departing from the true spirit and nature of the present invention.

The earplug assemblies' 100 of FIGS. 23-31 may include a two-part outer enclosure device 125, as best viewed in FIG. 33. This outer enclosure protects the user's hair or the like from entangling in the rotating reel mechanisms 102, 102′. If such an outer enclosure is applied, the enclosure itself will then be removably mounted to a mount surface of the hardhat 21 in the manner similar to that described heretofore.

Similar to the previous embodiments of FIGS. 1-22, the reeled mechanism embodiments of FIGS. 23-31 include a mounting assembly 128 that cooperates with the mount support portion 105 and a surface of the hardhat 21 to removably mount the protective housing 103 thereto. In one particular configuration, as shown in FIG. 31, the mounting assembly 100 can be a simple as double sided adhesive tape or VELCRO®. In other embodiments, the mounting assembly may be provided by other common adhesives and fasteners. In yet other mounting assembly configurations, as shown in FIG. 29, the housing 103 may incorporate one or more integrated mounting eyelets 126 or the like. These eyelets 126 enabled threaded mounting to fabric-based headwear or the like such as a visor or baseball-style caps.

Turning now to FIG. 34, another earplug assembly 130 of the present invention is shown having two earplug devices 27, 27′ mounted to one another via the opposed ends of a tether 28. In accordance with the embodiment of the present invention, a clip mechanism 131 is included that is disposed between the two earplug devices 27, 27′ on the tether 28. Accordingly, this embodiment enables the user to clip the earplug assembly 130 to anything that the clip mechanism can be removably mounted to.

Although the present invention has been described in connection with the preferred form of practicing it and modifications thereto, those of ordinary skill in the art will understand that many other modifications can be made thereto within the scope of the claims that follow. Accordingly, it is not intended that the scope of the invention in any way be limited by the above description, but instead be determined entirely by reference to the claims that follow.

What is claimed is:

1. A retractable earplug assembly for a hardhat including a crown portion having an interior facing wall, and a bottom edge portion thereof configured to extend generally equatorially around a forehead of a user when the hardhat is retained on the user's head, said earplug assembly including:
an elongated protective housing defining an interior channel extending generally from one end of the housing to an opposite end thereof, said housing further defining an elongated guide portion, extending substantially adjacent the interior channel, and an elongated mount support portion;
a mounting assembly configured to cooperate with the support portion and the interior facing wall of the crown portion to removably mount the protective housing thereto;
a guide base slideably cooperating with said guide portion for movement thereof along a fixed path in a direction generally longitudinally along substantially adjacent the interior channel between a first position and a second position;
a first earplug device; and
an elongated flexible first tether having a distal portion thereof mounted to the first earplug device, and a proximal portion thereof mounted to said guide base such that when said guide base moves along the fixed path between said first position and said second position, said first tether and said first earplug device move between a retracted condition and an extended condition, enabling usable access to the first earplug device.

2. The earplug assembly according to claim 1, wherein
said mounting assembly comprises a VELCRO®-type fastener material.

3. The earplug assembly according to claim 1, wherein
said guide portion and said guide base cooperate to form a zipper-type mechanism.

4. The earplug assembly according to claim 3, wherein
said protective housing is composed of a flexible sheet material.

5. The earplug assembly according to claim 1, wherein
said housing defines an exit opening, extending into said interior channel, for sliding receipt of said first tether therethrough between the first position and the second position.

6. The earplug assembly according to claim 1, wherein said housing is configured to cooperate with the hardhat crown portion such that when removably mounted thereto, said opposite end is position such that said first earplug device is positioned proximate the bottom edge portion of the hardhat when in the retracted condition.

7. The earplug assembly according to claim 6, wherein
said protective housing is comprised of a moldable polymer material, said support portion having a curvature generally conforming to that of the interior wall of the hardhat crown portion that the earplug assembly is to be mounted to.

8. The earplug assembly according to claim 7, wherein
said support portion is configured to seat in an elongated OEM channel formed in the interior wall of the hardhat.

9. The earplug assembly according to claim 8, wherein
said mounting assembly includes a plurality of spaced tab devices configured for friction fit mounting of the protective housing to the crown portion interior wall.

10. The earplug assembly according to claim 9, wherein
said tab devices are disposed in pairs on opposite sides of said OEM channel at two or more spaced locations longitudinally therealong.

11. The earplug assembly according to claim 9, wherein
each said tab device includes a tab member upstanding from the hardhat interior wall, and a corresponding engagement indent formed in aligned locations along the support portion, and configured for aligned engagement with a corresponding tab member when said protective housing is mounted to the crown portion.

12. The earplug assembly according to claim 7, wherein
the housing guide portion defines a pair of opposed elongated flange portions forming an elongated slot extending along and in communication with the interior channel, and
said guide base slideably cooperates with the opposed flange portions for sliding movement between the first position and the second position.

13. The earplug assembly according to claim 1, further including:
a covered parking structure cooperating with the hardhat crown portion to form a covered parking receptacle formed and dimensioned for receipt of the first earplug device therein in a manner substantially concealing the first earplug device under the parking structure when in the retracted position.

14. The earplug assembly according to claim 1, further including:
a second earplug device; and
an elongated flexible second tether having a distal portion thereof mounted to the second earplug device, and a proximal portion thereof mounted to said guide base such that when said guide base moves along the fixed path between said first position and said second position, said first and second tether and said first and second earplug device move simultaneously between the retracted condition and the extended condition, enabling usable access to the first and second earplug device.

15. A hardhat assembly comprising:
a hardhat including a crown portion having an interior facing wall, and a bottom edge portion thereof configured to extend generally equatorially around a forehead of a user when the hardhat is retained on the user's head;
an elongated protective housing extending along the interior facing wall, and defining an interior channel extending generally from one end of the housing to an opposite end thereof, said housing further defining an elongated guide portion, extending substantially adjacent the interior channel;
a guide base slideably cooperating with said guide portion for movement thereof along a fixed path in a direction generally longitudinally along substantially adjacent the interior channel between a first position and a second position;
an first earplug device; and
an elongated flexible first tether having a distal portion thereof mounted to the first earplug device, and a proximal portion thereof mounted to said guide base such that when said guide base moves along the fixed path between said first position and said second position, said first tether and said first earplug device move between a retracted condition and an extended condition, enabling usable access to the first earplug device.

16. The hardhat assembly according to claim 15, wherein
said protective housing is integrally molded with the interior facing wall of the crown portion.

17. The hardhat assembly according to claim 15, wherein
said protective housing includes a mount support portion and a mounting assembly configured to cooperate with the support portion and the interior facing wall of the crown portion to removably mount the protective housing thereto.

18. The hardhat assembly according to claim 17, wherein
said mounting assembly comprises a VELCRO®-type fastener material.

19. The hardhat assembly according to claim 14, wherein
said guide portion and said guide base cooperate to form a zipper-type mechanism.

20. The hardhat assembly according to claim 15, wherein
said housing defines an exit opening, extending into said interior channel, for sliding receipt of said first tether therethrough between the first position and the second position.

21. The hardhat assembly according to claim 15, wherein
said opposite end of said housing is position such that said first earplug device is positioned proximate the bottom edge portion of the hardhat when in the retracted condition.

22. The hardhat assembly according to claim 17, wherein
said protective housing is comprised of a moldable polymer material, said support portion having a curvature generally conforming to that of the interior wall of the hardhat crown portion that the hardhat assembly is to be mounted to.

23. The hardhat assembly according to claim 22, wherein
said support portion is configured to seat in an elongated OEM channel formed in the interior wall of the hardhat assembly.

24. The hardhat assembly according to claim 22, wherein
said mounting assembly includes a plurality of spaced tab devices configured for friction fit mounting of the protective housing to the crown portion interior wall.

25. The hardhat assembly according to claim 23, wherein
said tab devices are disposed in pairs on opposite sides of said OEM channel at two or more spaced locations longitudinally therealong.

26. The hardhat assembly according to claim 24, wherein
each said tab device includes a tab member upstanding from the hardhat interior wall, and a corresponding engagement indent formed in aligned locations along the support portion, and configured for aligned engagement with a corresponding tab member when said protective housing is mounted to the crown portion.

27. The hardhat assembly according to claim 22, wherein
the housing guide portion defines a pair of opposed elongated flange portions forming an elongated slot extending along and in communication with the interior channel, and
said guide base slideably cooperates with the opposed flange portions for sliding movement between the first position and the second position.

28. The hardhat assembly according to claim 15, further including:
a covered parking structure cooperating with the hardhat crown portion to form a covered parking receptacle formed and dimensioned for receipt of the first earplug device therein in a manner substantially concealing the first earplug device under the parking structure when in the retracted position.

29. The hardhat assembly according to claim 15, further including:
a second earplug device; and
an elongated flexible second tether having a distal portion thereof mounted to the second earplug device, and a proximal portion thereof mounted to said guide base such that when said guide base moves along the fixed path between said first position and said second position, said first and second tether and said first and second earplug device move simultaneously between the retracted condition and the extended condition, enabling usable access to the first and second earplug devices.

30. A hardhat assembly comprising:
a hardhat including a crown portion having an interior facing wall, and a bottom edge portion thereof configured to extend generally equatorially around a forehead of a user when the hardhat is retained on the user's head;
a retractable earplug assembly including:
a protective housing defining a mount support portion;
tether support structure;
a first earplug device;
an elongated flexible first tether having a distal portion thereof mounted to the first earplug device, and a proximal portion thereof movably cooperating with the tether support structure between a first position and a second position,
wherein, in said first position of the first tether proximal portion, the elongated first tether retractably cooperates with the tether support structure such that the distal portion of the flexible first tether and the first earplug device are positioned proximate to said protective housing, in a retracted condition, and
wherein, in said second position, said distal portion of the flexible first tether and the first earplug device can be repositioned away from the protective housing by a selected distance, enabling usable access to the first earplug device, in an extended condition; and
a mounting assembly configured to cooperate with the mount support portion and a surface of the hardhat to removably mount the protective housing thereto.

31. The hardhat assembly according to claim 30, wherein
said tether support structure includes a first reel mechanism having a first spool rotating about a first spool axis, said proximal portion of the first tether cooperating with said first spool for rotation about said first spool axis between the first position and second position.

32. The hardhat assembly according to claim 31, wherein
said first reel mechanism includes a first torsion spring cooperating with the first spool to bias the spool toward the first position, and the first tether and the first earplug assembly toward the retracted condition.

33. The hardhat assembly according to claim 32, wherein
said first reel mechanism is configured to spool the first tether toward the extended condition, in a stepped manner, preventing rotation of the first spool toward the first position and rewinding of the first tether toward the retracted condition.

34. The hardhat assembly according to claim 33, wherein
said first reel mechanism includes a release device, selectively engagable to enable the first torsion spring to rotate the first spool toward the first position.

35. The hardhat assembly according to claim 31, further including:
a second earplug device; and
an elongated flexible second tether having a distal portion thereof mounted to the second earplug device, and a proximal portion thereof cooperating with said first spool for rotation about said first spool axis between the first position and second position, such that said first tether and said second tether simultaneously move between the retracted condition and the extended condition.

36. The hardhat assembly according to claim 35, wherein
said first tether exits through a first opening on one side of said housing, and said second said second tether exits through a second opening on an opposite side of said housing.

37. The hardhat assembly according to claim 36, wherein
said housing defines a pair of opposed neck portions each protruding radially away from the first spool axis in substantially opposite directions thereof, one said neck portion defining the first opening and the other said neck portion defining the second opening.

38. The hardhat assembly according to claim 31, further including:
a second earplug device; and
an elongated flexible second tether having a distal portion thereof mounted to the second earplug device;
said tether support structure further including a second reel mechanism having a second spool rotating about a second spool axis, said second tether having a proximal portion cooperating with said second spool for rotation about said second spool axis between a respective first position and a respective second position,
wherein, in said first position of the second tether proximal portion, the elongated second tether retractably and rotatably cooperates with second spool such that the distal portion of the flexible first tether and the first earplug device are positioned proximate to said protective housing, in a retracted condition, and
wherein, in said second position, said distal portion of the flexible first tether and the first earplug device can be repositioned away from the protective housing by a selected distance, enabling usable access to the second earplug device, in an extended condition.

39. The hardhat assembly according to claim 38, wherein
said protective housing includes a first housing portion, supporting said first reel mechanism, and a second housing portion, supporting said second reel mechanism thereon,
said first housing portion and said second housing portion are configured to cooperatively articulate relative to one another.

40. The hardhat assembly according to claim 39, further including:
a hinge member coupling the first housing portion to the second housing portion in a hinged manner.

41. The hardhat assembly according to claim 30, wherein
said protective housing is elongated, and
said tether support structure cooperates with the protective housing to define an interior channel extending generally from one end of the housing to an opposite end thereof, said housing further defining an elongated guide portion, extending substantially adjacent the interior channel, and an elongated mount support portion, said tether support structure further including a guide base, coupled to the first tether proximal portion, and slideably cooperating with said guide portion for movement thereof along a fixed path in a direction generally longitudinally along substantially adjacent the interior channel between the first position and the second position, causing movement of the first tether between the retracted condition and the extended condition.
